(19) United States
(12) Patent Application Publication
(10) Pub. No.: US 2017/0121235 A1
(43) Pub. Date: May 4, 2017

(54) CONTROLLING SYSTEM AND METHOD FOR MAKING COMPOST

(71) Applicant: INSTITUTE FOR INFORMATION INDUSTRY, TAIPEI (TW)

(72) Inventor: Chien-Der Lin, Kaohsiung City (TW)

(21) Appl. No.: 14/953,501

(22) Filed: Nov. 30, 2015

(30) Foreign Application Priority Data
Nov. 4, 2015 (TW) 104136354

(51) Int. Cl. C05F 17/02; G05D 9/12; C05F 17/00; C12M 1/34; C12M 1/00; C05F 9/02; G05D 23/19; G05D 21/02

(52) U.S. Cl. CPC C05F 17/0276; G05D 23/1919; G05D 9/12; G05D 21/02; C12M 41/12; C12M 41/14; C05F 9/02; C05F 17/027; C05F 17/0258; C05F 17/0054; C05F 17/0063

(57) ABSTRACT

A method and a system for making compost are provided. The method includes following steps. First, a temperature of a compost controlling system is obtained. A heater module is turned on in a first mode. If the temperature is higher than a first predetermined temperature in the first mode, it switches into a second mode and the heater module is turned off. If the temperature is higher than a second predetermined temperature which is higher than the second predetermined temperature in the second mode, it switches into a third mode. If the temperature is lower than a third predetermined which is lower than the second predetermined temperature in the third mode, a compost completion message is generated. Accordingly, a probability of making the compost successfully is increased.

### RELATED APPLICATIONS

This application claims priority to Taiwan Application Serial Number 104136354 filed Nov. 4, 2015, which is herein incorporated by reference.

### BACKGROUND

Field

The present disclosure relates to a compost controlling system and a method. More particularly, the present disclosure relates to a system and a method for controlling the process of making compost according to several environment factors.

Description of Related Art

Fertile soil must have enough decomposed organic matter (also referred to humus) to provide an appropriate environment for corps. The purpose for making compost is to decompose organic matter, in which “decompose” means the extent of the decomposition has reached a certain level suitable for the corps instead the organic matter is completely decomposed. In general, compost is dark brown, delicate, soft and has a dirt aroma. During the process of making compost, factors such as temperature, oxygen concentration, stirring time, and standing time need to be controlled. For example, Taiwan patent publication number 201302663 disclosed a pre-treatment equipment for composting of agricultural waste in which the agricultural waste is crushed and dehydrated. Taiwan utility model number M326015 discloses a fast composting device, in which a container has a heat isolation case, and the process of composting is kept at a high temperature to perform aerobic fermentation by the isolation case and air circulation functions of a ventilation device. However, these prior arts do not consider and control all the factors. Therefore, it is an issue concerned in the art that how the factors are controlled in a systematic way to improve the possibility of making compost successfully.

### SUMMARY

Embodiments of the disclosure provides a compost controlling system including a container, a temperature sensor, a heater, and a controlling circuit. The temperature sensor is disposed on the container, and configured to detect a temperature corresponding to the container. The controlling circuit is electrically coupled to the temperature sensor and the heater. The controlling circuit turns on the heater and determine whether the temperature is higher than a first predetermined temperature in a first mode. If the temperature is higher than the first predetermined temperature in the first mode, the controlling circuit enters a second mode, turns off the heater, and determines whether the temperature is higher than a second predetermined temperature. The second predetermined temperature is higher than the first predetermined temperature. If the temperature is higher than the second predetermined temperature in the second mode, the controlling circuit enters a third mode and determines whether the temperature is lower than a third predetermined temperature. The third predetermined temperature is lower than the second predetermined temperature. If determining that the temperature is lower than the third predetermined temperature, the controlling circuit generates a compost completion message.

In an embodiment, the controlling circuit determines whether the temperature is lower than the first predetermined temperature in the second mode. If the temperature is lower than the first predetermined temperature in the second mode, the controlling circuit turns on the heater.

In an embodiment, the compost controlling system further includes a stirring module disposed in the container. If the controlling circuit determines that a first predetermined time is elapsed or the temperature is lower than a fourth predetermined temperature in the third mode, the controlling circuit turns on the stirring module. The fourth predetermined temperature is lower than the second predetermined temperature and higher than the third predetermined temperature.

In an embodiment, after turning on the stirring module, the controlling circuit determines whether the temperature is lower than a fifth predetermined temperature. The fifth predetermined temperature is lower than the fourth predetermined temperature and higher than the third predetermined temperature. If determining that the temperature is lower than the fifth predetermined temperature, the controlling circuit turns off the stirring module.

In an embodiment, the compost controlling system further includes a humidity sensor and a humidifier which are disposed on the container and electrically coupled to the controlling circuit. The humidity sensor detects a humidity corresponding to the container. The controlling circuit determines whether the humidity is lower than a predetermined humidity in the first mode and the second mode. If determining that the humidity is lower than the predetermined humidity, the controlling circuit turns on the humidifier.

In an embodiment, the compost controlling system further includes a liquid level sensor and a liquid fertilizer discharging device which are disposed on the container and electrically coupled to the controlling circuit. The liquid level sensor detects a liquid level corresponding to the container. The controlling circuit determines whether the liquid level is higher than a predetermined liquid level in the third mode. If determining that the liquid level is higher than the predetermined liquid level, the controlling circuit turns on the liquid fertilizer discharging device.

In an embodiment, the compost controlling system further includes an oxygen sensor and a gas providing device which are disposed on the container and electrically coupled to the controlling circuit. The oxygen sensor detects an oxygen value corresponding to the container. The controlling circuit determines whether the oxygen value is less than a predetermined oxygen value in the second mode. If determining that the oxygen value is less than the predetermined oxygen value, the controlling circuit turns on the gas providing device.

In an embodiment, the compost controlling system further includes a weight sensor disposed on the container and electrically coupled to the controlling circuit. The weight detects a weight value corresponding to the container.

In an embodiment, the compost controlling system further includes a display device disposed on the container and electrically coupled to the controlling circuit. When the controlling circuit is in the second mode, the controlling circuit records time elapsed in the first mode and accordingly calculates estimated completion time, and displays the estimated completion time on the display device.

In an embodiment, when the controlling circuit is in the third mode, the controlling circuit records time elapsed in the first mode and the second mode, and accordingly recalculates and updates the estimated completion time. The controlling circuit displays the updated estimated completion time on the display device.

Embodiments of the disclosure provides a compost controlling method including following steps. First, a temperature corresponding to the compost controlling system is obtained. A heater is turned on, and whether the temperature is higher than a first predetermined temperature is determined in a first mode. If the temperature is higher than the first predetermined temperature in the first mode, a second mode is entered, the heater is turned off, and whether the temperature is higher than a second predetermined temperature is determined. The second predetermined temperature is higher the first predetermined temperature. If the temperature is higher than the second predetermined temperature in the second mode, a third mode is entered, and whether the temperature is lower than a third predetermined temperature is determined. The third predetermined temperature is lower than the second predetermined temperature. A compost completion message is generated if determining that the temperature is lower than the third predetermined temperature in the third mode.

In an embodiment whether the temperature is lower than the first predetermined temperature is determined in the second mode. The heater is turned on if the temperature is lower than the first predetermined temperature in the second mode.

In an embodiment, a stirring module is turned on if determining that first predetermined time has elapsed or the temperature is lower than a fourth predetermined temperature in the third mode. The fourth predetermined temperature is lower than the second predetermined temperature and higher than the third predetermined temperature.

In an embodiment, whether the temperature is lower than a fifth predetermined temperature is determined after turning on the stirring module. The fifth predetermined temperature is lower than the fourth predetermined temperature and higher than the third predetermined temperature. The stirring module is turned off if determining that the temperature is lower than the fifth predetermined temperature.

In an embodiment, a humidity corresponding to the compost controlling system is obtained. Whether the humidity is lower than a predetermined humidity is determined in the first mode and the second mode. A humidifier is turned on if determining that the humidity is lower than the predetermined humidity.

In an embodiment, a liquid level corresponding to the compost controlling system is obtained. Whether the liquid level is higher than a predetermined liquid level is determined in the third mode. A liquid fertilizer discharging device is turned on if determining that the liquid level is higher than the predetermined liquid level.

In an embodiment, an oxygen value corresponding to the compost controlling system is obtained. Whether the oxygen value is less than a predetermined oxygen value is determined in the second mode. A gas providing device is turned on if determining that the oxygen value is less than the predetermined oxygen value.

In an embodiment, a weight value corresponding to the compost controlling system is obtained.

In an embodiment, time elapsed in the first mode is recoded when entering the second mode. Estimated completion time is calculated accordingly, and is displayed on a display device.

In an embodiment, time elapsed in the first mode and the second mode is recorded when entering the third mode. The estimated completion time is updated accordingly, and the updated estimated completion time is displayed on the display device.

### BRIEF DESCRIPTION OF THE DRAWINGS

The disclosure can be more fully understood by reading the following detailed description of the embodiment, with reference made to the accompanying drawings as follows:

FIG. 1 is a schematic diagram illustrating a compost controlling system according to an embodiment.

FIG. 2 is a diagram illustrating a temperature curve for making compost according to an embodiment.

FIG. 3 is a diagram illustrating the temperature curve for making compost according to another embodiment.

FIG. 4A and FIG. 4B are diagrams illustrating a compost controlling method according to an embodiment.

### DETAILED DESCRIPTION

Specific embodiments of the present disclosure are further described in detail below with reference to the accompanying drawings, however, the embodiments described are not intended to limit the present disclosure and it is not intended for the description of operation to limit the order of implementation. Moreover, any device with equivalent functions that is produced from a structure formed by a recombination of elements shall fall within the scope of the present disclosure. Additionally, the drawings are only illustrative and are not drawn to actual size.

The using of “first”, “second”, etc. in the specification should be understood for identify units or data described by the same terminology, but are not referred to particular order or sequence.

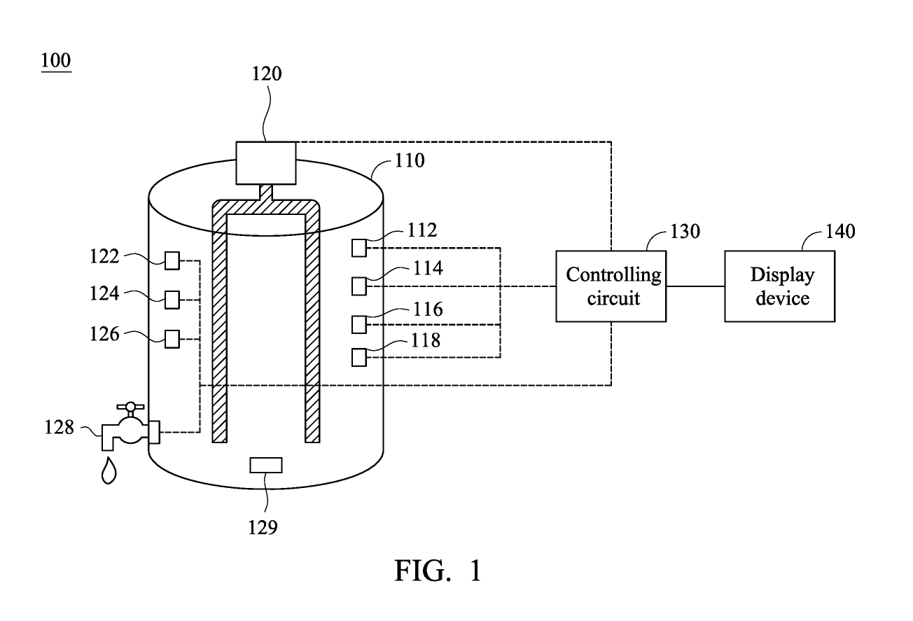

FIG. 1 is a schematic diagram illustrating a compost controlling system according to an embodiment. Referring to FIG. 1, a compost controlling system 100 mainly includes a container 110, a temperature sensor 112, a heater 122 and a controlling circuit 130. In other embodiments, the compost controlling system may further include a humidity sensor 114, an oxygen sensor 116, a liquid level sensor 118, a stirring module 120, a humidifier 124, a gas providing device 126 and a liquid fertilizer discharging device 128. The container 110 is, for example, a bucket for storing compost. But the material, shape, or size of the container 110 is not limited in the disclosure. In the embodiment, the temperature sensor 112, the humidity sensor 114, the oxygen sensor 116, the liquid level sensor 118, the stirring module 120, the heater 122, the humidifier 124, the gas providing device 126 and the liquid fertilizer discharging device 128 is disposed on the container 110, but the disposition locations of these units are limited in the disclosure. In addition, these units are electrically coupled to the controlling circuit 130 in a wire or wireless way. The controlling circuit 130 may be implemented as any kind of processor or programmable circuit. In the embodiment, the controlling circuit 130 controls the compost controlling system 100 according to sensed temperature, humidity and oxygen saturation, and displays messages on the display device 140. Several embodiment will be provided below.

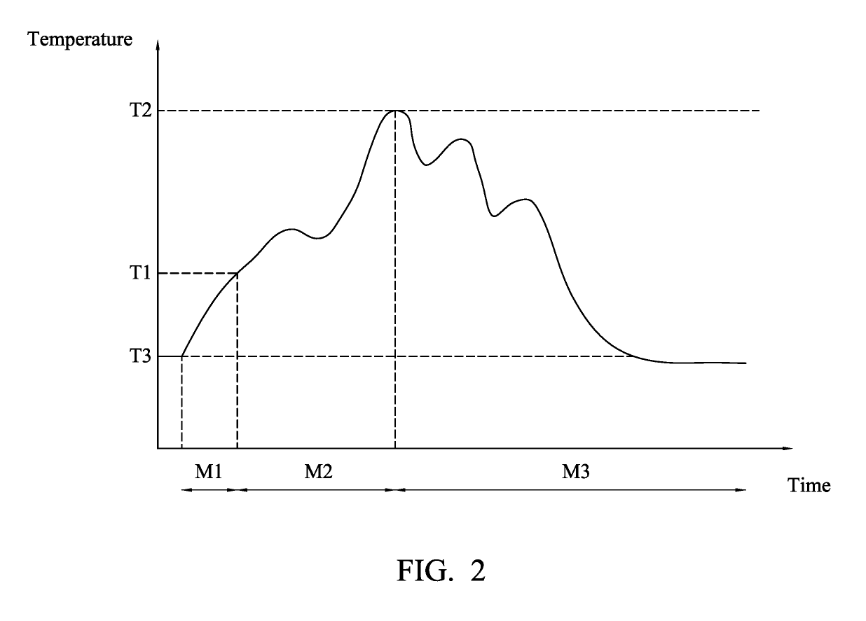

FIG. 2 is a diagram illustrating a temperature curve for making compost according to an embodiment. Referring to FIG. 1 and FIG. 2, first, the temperature sensor 112 detests a temperature corresponding to the container 110, and transmits the temperature to the controlling circuit 130. In an initialization stage, the controlling circuit 130 is in a first mode M1, and the container 110 is under a room temperature. In the mode M1, the controlling circuit 130 turns on the heater 122, and therefore the temperature of the container 110 is progressively increased. The controlling circuit 130 also determines whether the temperature obtained from the temperature sensor 112 is higher than a first predetermined temperature T1. The first predetermined temperature T1 is, for example, ranged from 40 degrees Celsius to 50 degree Celsius. The first mode M1 is configured to increase the activity of the microorganisms in the container 110, and at this moment, some of the microorganisms have started to decompose organic matters.

If the temperature is higher than the first predetermined temperature T1 in the first mode M1, the controlling circuit 130 will be set as a second mode M2 (also referred to the controlling circuit 130 entering the second mode M2). In the second mode M2, the controlling circuit 130 turns off the heater 122, and determines whether the temperature obtained from temperature sensor 112 is higher than the second predetermined temperature T2 which is higher than the first predetermined temperature T1. For example, the second predetermined temperature T2 is ranged from 60 degrees Celsius to 80 degrees Celsius. In the second mode M2, the microorganisms have stated to decompose massive organic matters. The decomposition generates lots of heat, and thus the heater 122 is turned off. In some embodiment, if the sensed temperature is lower than the first predetermined temperature T1 in the second mode M2 due to some external factors, then the controlling circuit 130 will again turns on the heater 122 until the temperature is higher than the first predetermined temperature T1. Since the decomposition generates lots of heat in the second mode M2, some prior arts do not have the mechanism of re-turning on the heater 122. However, in practical scenarios, the temperature of the compost may drop due to the user opening the container or stirring the compost, or due to the cold air outside. In the embodiment, the mechanism of re-turning on the heater 122 increases the probability of making the compost successfully, which is an advantage of the disclosure.

In some embodiments, the controlling circuit 130 controls the heater 122 based on a fuzzy inference control, but the disclosure is not limited thereto. The controlling circuit 130 may apply a proportional-integral-derivative (PID) control in other embodiments.

If the sensed temperature is higher than the second predetermined temperature T2 in the second mode M2, the controlling circuit 130 will be set as a third mode M3 (or referred to the controlling circuit 130 entering the third mode). In the third mode M3, the temperature drops progressively, and the controlling circuit 130 determines whether the sensed temperature is lower than a third predetermined temperature T3 which is lower than the second predetermined temperature T2. For example, the third predetermined temperature T3 is near to the room temperature (e.g. 25 degrees Celsius). If determining that the temperature is lower than the third predetermined temperature T3, the controlling circuit 130 will generates a compost completion message, and display the compost completion message on the display device 140. The compost completion message is used to inform the user about the making of the compost being done. The compost completion message may be implemented as words, images, symbols, etc. Moreover, the content of the compost completion message is not limited in the disclosure. In practice, the compost no longer generates heat after the decomposition is finished, and therefore the temperature of the container may drop because there is no heat isolation means in the container in some embodiments.

The controlling circuit 130 has three modes, and only controls the heater 122 in the embodiments above. However, the three modes may be divided into more modes, and the stirring module 120, the humidifier 124, the gas providing device 126 and the liquid fertilizer discharging device 128 may be controlled as well in the embodiments below.

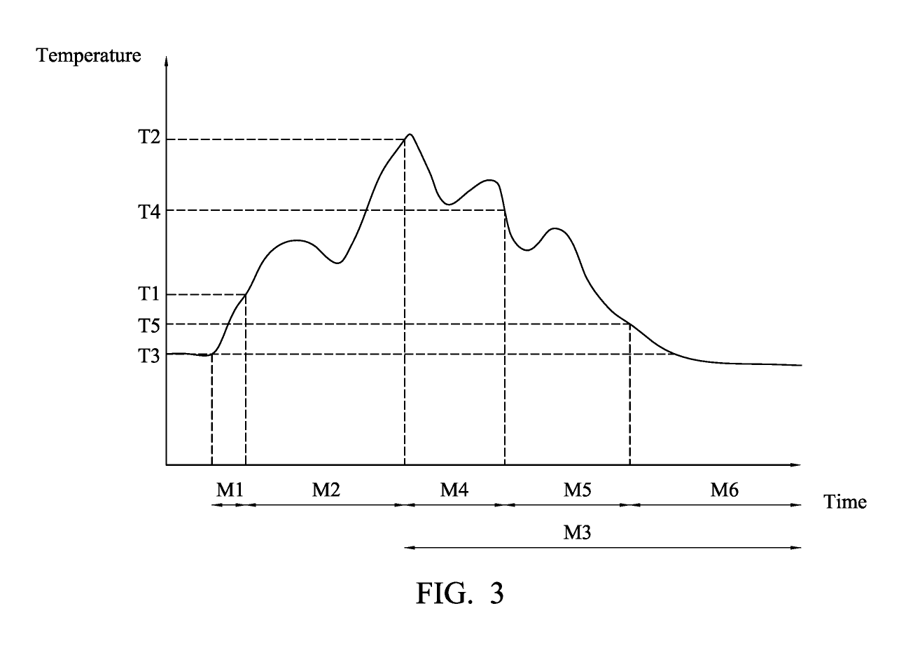

FIG. 3 is a diagram illustrating the temperature curve for making compost according to another embodiment. Referring to FIG. 1 and FIG. 3 together, the third mode M3 is further divided into a fourth mode M4, a fifth mode M5 and a sixth mode M6. In the fourth mode M4, the controlling circuit 130 determines whether first predetermined time is elapsed and/or the sensed temperature is lower than a fourth predetermined temperature T4. The first predetermined time is, for example, two or three days, and the purpose thereof is to let the compost stand. On the other hand, the fourth predetermined temperature T4 is lower than the second predetermined temperature T2 and higher than the third predetermined temperature T3. For example, the fourth predetermined temperature T4 is ranged from 55 degrees Celsius to 75 degrees Celsius. When the sensed temperature is lower than the fourth predetermined temperature T4, it means the process of the standing is finished. When the first predetermined time is elapsed and/or the sensed temperature is lower than the fourth predetermined temperature T4, the controlling circuit 130 enters the fifth mode M5, and turns on the stirring module 120 to stir the compost. In the fifth mode M5, the controlling circuit 130 determines whether the sensed temperature is lower than the fifth predetermined temperature T5 which is lower than the fourth predetermined temperature T4 and higher than the third predetermined temperature T3. For example, the fifth predetermined temperature is about 35 degrees Celsius. If the sensed temperature is lower than the fifth predetermined temperature T5, the controlling circuit 130 turns off the stirring module 120 and enters the sixth mode M6. In the sixth mode M6, when the sensed temperature is lower than the third predetermined temperature T3, it means the making of the compost is finished.

On the other hand, the controlling circuit 130 controls the humidity in the first mode M1 and the second mode M2. To be specific, the humidity sensor 114 detects a humidity corresponding to the container 110, and transmits the information of the humidity to the controlling circuit 130. The controlling circuit 130 determines whether the humidity is lower than a predetermined humidity (it varies depending on the types of the compost and the microorganisms, for example, it ranges from 50% to 80%). If the sensed humidity is lower than the predetermined humidity in the first mode M1 and the second mode M2, then the controlling circuit 130 turns on the humidifier 124. For example, the humidifier 124 increases the humidity of the compost through spraying water.

The controlling circuit 130 controls the concentration of the oxygen in the second mode M2 and the fourth mode M4. To be specific, the oxygen sensor 116 detects an oxygen value (e.g. the concentration of the oxygen) corresponding to the container 110, and transmits the oxygen value to the controlling circuit 130. The controlling circuit 130 determines whether the oxygen value is less than a predetermined oxygen value (it varies depending on the types of the compost and the microorganisms, for example, it is 20%). If the oxygen value is less than the predetermined oxygen value in the second mode M2 and the fourth mode M4, the controlling circuit 130 turns on the gas providing device 124. For example, the gas providing device 124 imports the external fresh air into the container 110.

The controlling circuit 130 controls a liquid level of a liquid fertilizer in the fourth mode M4 and the fifth mode M5. To be specific, the liquid level sensor 118 detects a liquid level (representing the level of the liquid fertilizer) corresponding to the container 110, and transmits the liquid level to the controlling circuit 130. The controlling circuit 130 determines whether the liquid level is higher than a predetermined liquid level (it varies depending on the shapes of the container 110, for example, it is two centimeter). If the controlling circuit 130 determines that the liquid level is higher than the predetermined liquid level in fourth mode M4 and the fifth mode M5, the controlling circuit 130 turns on the liquid fertilizer discharging device 128 so that the liquid fertilizer is discharged into outside of the container 110. In some embodiments, the compost controlling system 100 also has a liquid fertilizer collecting module for collecting the discharged liquid fertilizer.

The weight sensor 129 is used to detect a weight value corresponding to the container 110. For example, the weight value may represent how much compost is in the container 110. When the weight value is larger, it means the time to make the compost is required to be longer. In some embodiments, the first predetermined time is proportional to the weight value.

In some embodiments, the controlling circuit 130 also predicts when the making of the compost is finished, and displays the predicted time on the display device 140. To be specific, when the controlling circuit 130 enters the second mode M2, the controlling circuit 130 records time elapsed in the first mode M1, and accordingly calculates estimated completion time, and displays the estimated completion time on the display device 140. In addition, when the controlling circuit 130 enters the fourth mode M4 (i.e. entering the third mode M3), the controlling circuit 130 records time elapsed in the first mode M1 and the second mode M2, and re-calculates and updates the estimated completion time according to the recorded time information. The controlling circuit 130 displays the updated estimated completion time on the display device 140. In some embodiments, the controlling circuit 130 may calculates the estimated completion time according to the time elapsed in the first mode M1 and the second mode M2, and the weight value sensed by the weight sensor 129. Consequently, the user may know when the making of the compost is finished, and the estimated finished time is updated in real time during the composting procedure.

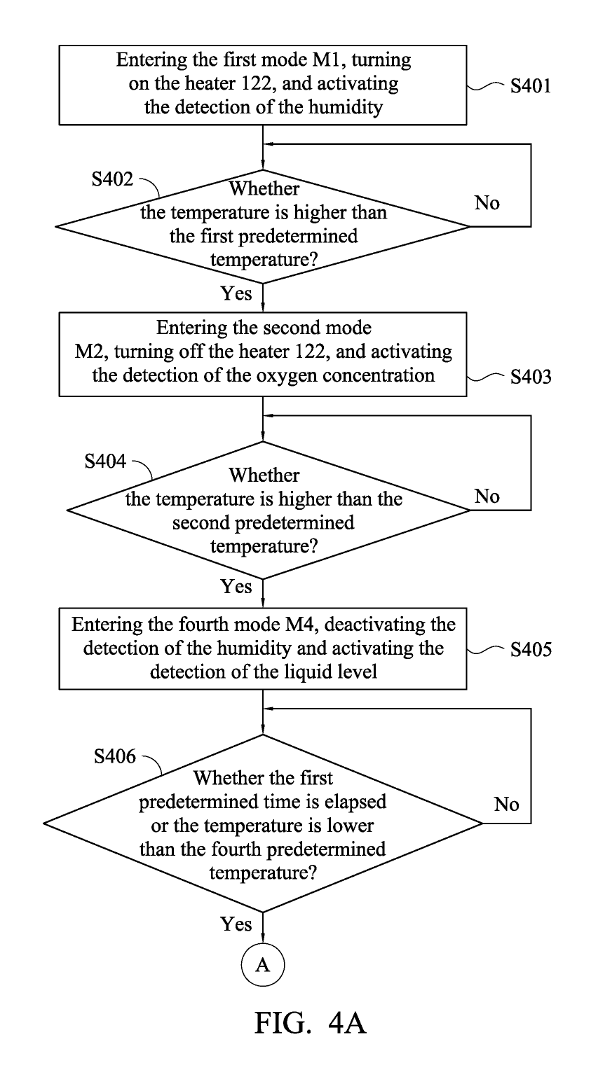

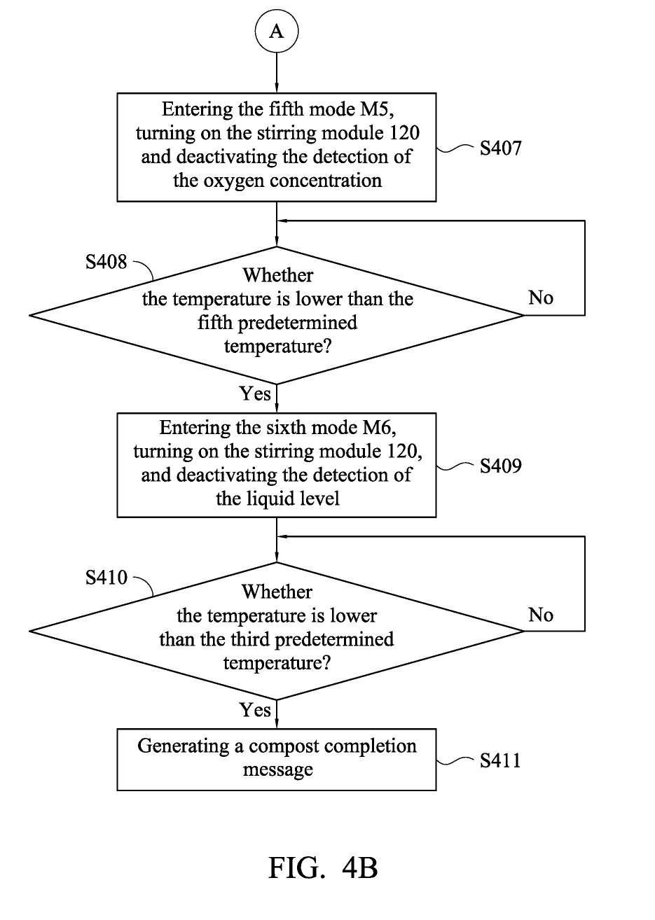

FIG. 4A and FIG. 4B are diagrams illustrating a compost controlling method according to an embodiment. Referring to FIG. 4A and FIG. 4B, the first mode M1 is entered, the heater 122 is turned on and the detection of the humidity is activated in a step S401. In a step S402, whether the temperature is higher than the first predetermined temperature is determined. If the temperature is higher than the first predetermined temperature, the second mode M2 is entered, the heater 122 is turned off, and the detection of the oxygen concentration is activated in a step S403. In a step S404, whether the temperature is higher than the second predetermined temperature is determined. If the temperature is higher than the second predetermined temperature, the fourth mode M4 is entered, the detection of the humidity is deactivated and the detection of the liquid level is activated in a step S405. In a step S406, whether the first predetermined time is elapsed or whether the temperature is lower than the fourth predetermined temperature is determined. If the determination result of the step S406 is affirmative, the fifth mode M5 is entered, the stirring module 120 is turned on and the detection of the oxygen concentration is deactivated in a step S407. In a step S408, whether the temperature is lower than the fifth predetermined temperature is determined. If the temperature is lower than the fifth predetermined temperature, the sixth mode M6 is entered, the stirring module 120 is turned off, and the detection of the liquid level is deactivated in a step S409. In a step S410, whether the temperature is lower than the third predetermined temperature is determined. If the temperature is lower than the third predetermined temperature, the compost completion message is generated in a step S411.

However, all the steps in FIG. 4A and FIG. 4B have been described in detail above, and therefore they will not be repeated. Note that the steps in FIG. 4A and FIG. 4B can be implemented as program codes or circuits, and the disclosure is not limited thereto. In addition, the method in FIG. 4A and FIG. 4B can be performed with the aforementioned embodiments, or can be performed independently. In other words, other steps may be inserted between the steps of the FIG. 4A and FIG. 4B.

Although the present disclosure has been described in considerable detail with reference to certain embodiments thereof, other embodiments are possible. Therefore, the spirit and scope of the appended claims should not be limited to the description of the embodiments contained herein. It will be apparent to those skilled in the art that various modifications and variations can be made to the structure of the present disclosure without departing from the scope or spirit of the disclosure. In view of the foregoing, it is intended that the present disclosure cover modifications and variations of this disclosure provided they fall within the scope of the following claims.

What is claimed is:

1. A compost controlling system, comprising:
a container;
a temperature sensor, disposed on the container, and configured to detect a temperature corresponding to the container;
a heater, disposed on the container; and
a controlling circuit, electrically coupled to the temperature sensor and the heater, and configured to turn on the heater and determine whether the temperature is higher than a first predetermined temperature in a first mode,
wherein if the temperature is higher than the first predetermined temperature in the first mode, the controlling circuit enters a second mode, turns off the heater, and determines whether the temperature is higher than a second predetermined temperature, wherein the second predetermined temperature is higher than the first predetermined temperature,
if the temperature is higher than the second predetermined temperature in the second mode, the controlling circuit enters a third mode, and determines whether the temperature is lower than a third predetermined temperature, wherein the third predetermined temperature is lower than the second predetermined temperature, and
if determining that the temperature is lower than the third predetermined temperature, the controlling circuit generates a compost completion message.

2. The compost controlling system of claim 1, wherein the controlling circuit determines whether the temperature is lower than the first predetermined temperature in the second mode, and
if the temperature is lower than the first predetermined temperature in the second mode, the controlling circuit turns on the heater.

3. The compost controlling system of claim 1, further comprising:
a stirring module, disposed in the container,
wherein if the controlling circuit determines that a first predetermined time is elapsed or the temperature is lower than a fourth predetermined temperature in the third mode, the controlling circuit turns on the stirring module, wherein the fourth predetermined temperature is lower than the second predetermined temperature and higher than the third predetermined temperature.

4. The compost controlling system of claim 3, wherein after turning on the stirring module, the controlling circuit determines whether the temperature is lower than a fifth predetermined temperature, wherein the fifth predetermined temperature is lower than the fourth predetermined temperature and higher than the third predetermined temperature, and
if determining that the temperature is lower than the fifth predetermined temperature, the controlling circuit turns off the stirring module.

5. The compost controlling system of claim 1, further comprising:
a humidity sensor, disposed on the container and electrically coupled to the controlling circuit, and configured to detect a humidity corresponding to the container; and
a humidifier, disposed on the container and electrically coupled to the controlling circuit,
wherein the controlling circuit determines whether the humidity is lower than a predetermined humidity in the first mode and the second mode, and
if determining that the humidity is lower than the predetermined humidity, the controlling circuit turns on the humidifier.

6. The compost controlling system of claim 1, further comprising:
a liquid level sensor, disposed on the container and electrically coupled to the controlling circuit, and configured to detect a liquid level corresponding to the container; and
a liquid fertilizer discharging device, disposed on the container and electrically coupled to the controlling circuit,
wherein the controlling circuit determines whether the liquid level is higher than a predetermined liquid level in the third mode, and
if determining that the liquid level is higher than the predetermined liquid level, the controlling circuit turns on the liquid fertilizer discharging device.

7. The compost controlling system of claim 1, further comprising:
an oxygen sensor, disposed on the container and electrically coupled to the controlling circuit, and configured to detect an oxygen value corresponding to the container; and
a gas providing device, disposed on the container and electrically coupled to the controlling circuit,
wherein the controlling circuit determines whether the oxygen value is less than a predetermined oxygen value in the second mode, and
if determining that the oxygen value is less than the predetermined oxygen value, the controlling circuit turns on the gas providing device.

8. The compost controlling system of claim 1, further comprising:
a weight sensor, disposed on the container and electrically coupled to the controlling circuit, and configured to detect a weight value corresponding to the container.

9. The compost controlling system of claim 1, further comprising:
a display device, disposed on the container and electrically coupled to the controlling circuit,
wherein when the controlling circuit is in the second mode, the controlling circuit records time elapsed in the first mode and accordingly calculates estimated completion time, and displays the estimated completion time on the display device.

10. The compost controlling system of claim 9, wherein when the controlling circuit is in the third mode, the controlling circuit records time elapsed in the first mode and the second mode, and accordingly recalculates and updates the estimated completion time, and displays the updated estimated completion time on the display device.

11. A compost controlling method for a compost controlling system, the compost controlling method comprising:
obtaining a temperature corresponding to the compost controlling system;
turning on a heater, and determining whether the temperature is higher than a first predetermined temperature in a first mode;
if the temperature is higher than the first predetermined temperature in the first mode, entering a second mode, turning off the heater, and determining whether the temperature is higher than a second predetermined temperature, wherein the second predetermined temperature is higher the first predetermined temperature;
if the temperature is higher than the second predetermined temperature in the second mode, entering a third mode, and determining whether the temperature is lower than a third predetermined temperature, wherein the third predetermined temperature is lower than the second predetermined temperature; and
generating a compost completion message if determining that the temperature is lower than the third predetermined temperature in the third mode.

12. The compost controlling method of claim 11, further comprising:
determining whether the temperature is lower than the first predetermined temperature in the second mode; and
turning on the heater if the temperature is lower than the first predetermined temperature in the second mode.

13. The compost controlling method of claim 11, further comprising:
turning on a stirring module if determining that first predetermined time has elapsed or the temperature is lower than a fourth predetermined temperature in the third mode, wherein the fourth predetermined temperature is lower than the second predetermined temperature and higher than the third predetermined temperature.

14. The compost controlling method of claim 13, further comprising:
determining whether the temperature is lower than a fifth predetermined temperature after turning on the stirring module, wherein the fifth predetermined temperature is lower than the fourth predetermined temperature and higher than the third predetermined temperature; and
turning off the stirring module if determining that the temperature is lower than the fifth predetermined temperature.

15. The compost controlling method of claim 11, further comprising:
obtaining a humidity corresponding to the compost controlling system;
determining whether the humidity is lower than a predetermined humidity in the first mode and the second mode; and
turning on a humidifier if determining that the humidity is lower than the predetermined humidity.

16. The compost controlling method of claim 11, further comprising:
obtaining a liquid level corresponding to the compost controlling system;
determining whether the liquid level is higher than a predetermined liquid level in the third mode; and
turning on a liquid fertilizer discharging device if determining that the liquid level is higher than the predetermined liquid level.

17. The compost controlling method of claim 11, further comprising:
obtaining an oxygen value corresponding to the compost controlling system;
determining whether the oxygen value is less than a predetermined oxygen value in the second mode; and
turning on a gas providing device if determining that the oxygen value is less than the predetermined oxygen value.

18. The compost controlling method of claim 11, further comprising:
obtaining a weight value corresponding to the compost controlling system.

19. The compost controlling method of claim 11, further comprising:
recoding time elapsed in the first mode when entering the second mode, and accordingly calculating estimated completion time, and displaying the estimated completion time on a display device.

20. The compost controlling method of claim 19, further comprising:
recording time elapsed in the first mode and the second mode when entering the third mode, and accordingly recalculating and updating the estimated completion time, and displaying the updated estimated completion time on the display device.
